(19) United States
(12) Patent Application Publication
(10) Pub. No.: US 2020/0154982 A1
(43) Pub. Date: May 21, 2020

(54) ENDOSCOPE SYSTEM AND ENDOSCOPE CONVERTER

(71) Applicant: OLYMPUS CORPORATION, Tokyo (JP)

(72) Inventors: Hiroshi NIWA, Tokyo (JP); Yoshimine KOBAYASHI, Tokyo (JP)

(73) Assignee: OLYMPUS CORPORATION, Tokyo (JP)

(21) Appl. No.: 16/774,155

(22) Filed: Jan. 28, 2020

Related U.S. Application Data
(63) Continuation of application No. PCT/JP2018/017655, filed on May 7, 2018.

(30) Foreign Application Priority Data
Aug. 1, 2017 (JP) 2017-149430

(51) Int. Cl. A61B 1/00; A61B 1/012; A61B 1/06

(52) U.S. Cl. CPC A61B 1/00126; A61B 1/00117; A61B 1/0669; A61B 1/0125

(57) ABSTRACT

An endoscope system includes: a first endoscope including a first endoscope connector; a second endoscope including a second endoscope connector; an external apparatus including an apparatus-side receptacle to which the first endoscope connector and the second endoscope connector are connectable; and a converter including a plug connectable to the apparatus-side receptacle, a first receptacle to which the first endoscope connector is connectable, and a second receptacle to which the second endoscope connector is connectable.

### CROSS REFERENCE TO RELATED APPLICATION

This application is a continuation application of PCT/JP2018/017655 filed on May 7, 2018 and claims benefit of Japanese Application No. 2017-149430 filed in Japan on Aug. 1, 2017, the entire contents of which are incorporated herein by this reference.

### BACKGROUND OF INVENTION

### 1. Field of the Invention

The present invention relates to an endoscope system including a video system to which a plurality of endoscopes are connectable, and also to an endoscope converter.

### 2. Description of the Related Art

A mother-baby endoscope system has been conventionally put into a practical use, as an endoscope to be used for performing observation or treatment in a bile duct or a pancreatic duct. Such a mother-baby endoscope system includes a mother endoscope and a baby endoscope configured to be inserted into a treatment instrument insertion channel of the mother endoscope.

The mother endoscope of the mother-baby endoscope system is a side-view endoscope for duodenum, for example, and the baby endoscope is configured to be inserted into the treatment instrument insertion channel of the mother endoscope.

The mother-baby endoscope system is operated while combining bending operation, advancing/retreating operation, twisting operation, etc., of the baby endoscope protruded from the distal end of the mother endoscope into a body cavity and raising stand operation, bending operation, twisting operation, advancing/retreating operation etc., of the mother endoscope, to thereby cause only the baby endoscope to be inserted selectively into the bile duct or the pancreatic duct from the duodenal papilla to perform observation or treatment in the bile duct or the pancreatic duct.

Such a mother-baby endoscope system is used while observing insertion states of the mother endoscope and the baby endoscope using an X-ray image obtained by a contrast radiography under the X-ray fluoroscopy.

Various types of mother-baby endoscope systems have been proposed, for example, in Japanese Patent Application Laid-Open Publication No. 7-250813, and have been put into a practical use. The Japanese Patent Application Laid-Open Publication No. 7-250813 discloses an endoscope apparatus as a mother-baby endoscope system capable of effectively observing an observation object by displaying a plurality of observation images on one display apparatus.

### SUMMARY OF THE INVENTION

An endoscope system according to one aspect of the present invention includes: a first endoscope including a first endoscope connector; a second endoscope including a second endoscope connector; an external apparatus including an apparatus-side receptacle to which the first endoscope connector and the second endoscope connector are connectable; and a converter including a plug connectable to the apparatus-side receptacle, a first receptacle to which the first endoscope connector is connectable, and a second receptacle to which the second endoscope connector is connectable.

An endoscope converter according to another aspect of the present invention includes: a plug connectable to an apparatus-side receptacle of an external apparatus; a first receptacle to which a first endoscope connector included in a first endoscope is connectable; and a second receptacle to which a second endoscope connector included in a second endoscope is connectable.

An endoscope converter according to another aspect of the present invention includes: a first converter-side connector to which a first endoscope-side connector included in a first endoscope is connectable; a second converter-side connector to which a second endoscope-side connector included in a second endoscope is connectable; and a third converter-side connector connectable to an apparatus-side connector of an external apparatus.

### BRIEF DESCRIPTION OF THE DRAWINGS

FIG. 1 illustrates an endoscope system according to one aspect of the present invention and is an exterior view schematically illustrating a state where a baby endoscope is inserted into a mother endoscope.

FIG. 2 is a plan view illustrating a state where two endoscope connectors are to be connected to a video system through a converter according to the present invention.

FIG. 3 is a plan view illustrating a state where the two endoscope connectors are connected to the video system through the converter according to the present invention.

FIG. 4 is a schematic view illustrating an optical distributor in the converter and a light source in the video system according to the present invention.

FIG. 5 relates to a first modified example, and is a plan view illustrating a state where two endoscope connectors are connected to a video system through a converter.

FIG. 6 relates to a second modified example, and is a plan view illustrating a state where one of two endoscope connectors is an electrical connector that incorporates a light source and the two endoscope connectors are to be connected to a video system through a converter.

FIG. 7 relates to a third modified example, and is a plan view illustrating a state where one of two endoscope connectors is an electrical connector and the two endoscope connectors are to be connected to a video system through a converter.

FIG. 8 relates to a fourth modified example, and is a front view illustrating a state where a converter is connected to a video system.

FIG. 9 relates to a fifth modified example, and is a front view illustrating a state where a converter is connected to a video system.

FIG. 10 relates to a sixth modified example, and is a plan view illustrating a state where a monitor and two endoscope connectors are connected to a video system through a converter.

FIG. 11 relates to the sixth modified example, and is a plan view illustrating a state where the monitor and one of the endoscope connectors is connected to the video system through the converter.

FIG. 12 relates to a seventh modified example, and is a plan view illustrating a state where one of endoscope connectors is to be connected to a video system through a converter and the converter is provided with notification means.

FIG. 13 relates to the seventh modified example, and is a plan view illustrating a state where only one of the endoscope connectors is connected to the video system through the converter.

FIG. 14 relates to an eighth modified example, and is a plan view illustrating a state where two endoscope connectors and an electrical connector are to be connected respectively to a light source apparatus and a video processor through two converters.

FIG. 15 relates to a ninth modified example, and is a plan view illustrating a state where two endoscope connectors and an electrical connector are to be connected to a light source apparatus and a video processor through one converter.

### DETAILED DESCRIPTION OF THE PREFERRED EMBODIMENT(S)

Hereinafter, a preferred embodiment of the present invention will be described with reference to drawings. Note that a different scale size is used for each of the constituent elements in the drawings to be referred to in the description below, in order to allow each of the constituent elements to be illustrated in a recognizable size. The present invention is not limited only to the number, the shapes, ratio of the sizes of the constituent elements, and the relative positional relationship among the constituent elements illustrated in the drawings. In addition, there is a case where the up direction and the down direction as seen toward the paper surface are described respectively as the upper portion and the lower portion of a certain constituent element in the description below.

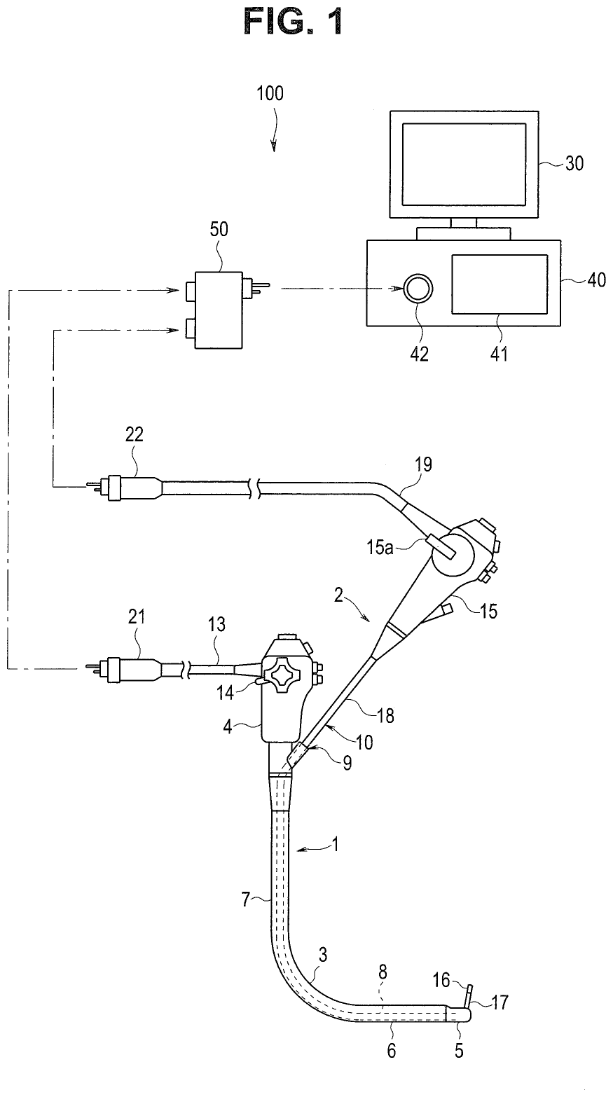

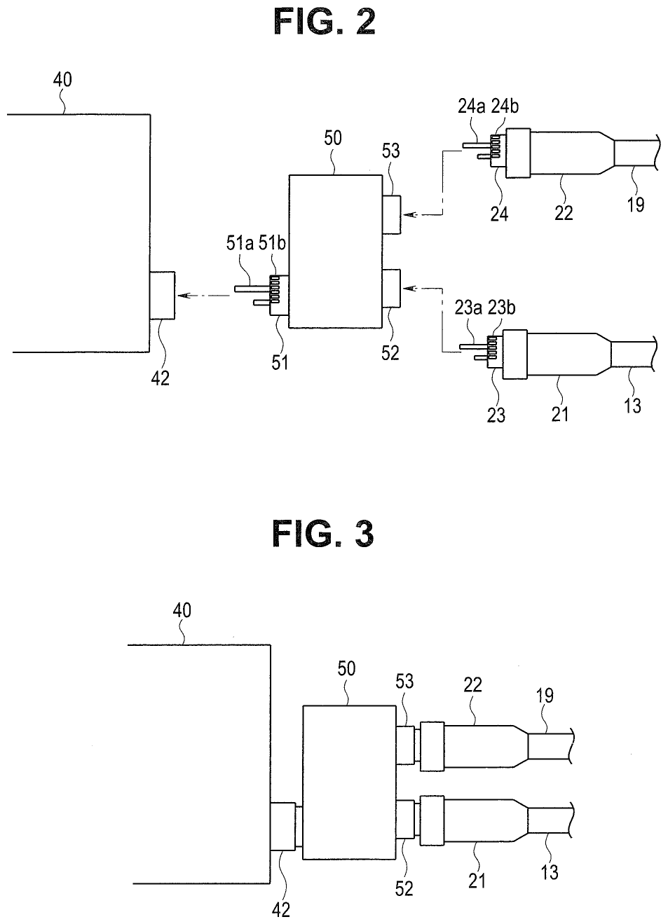

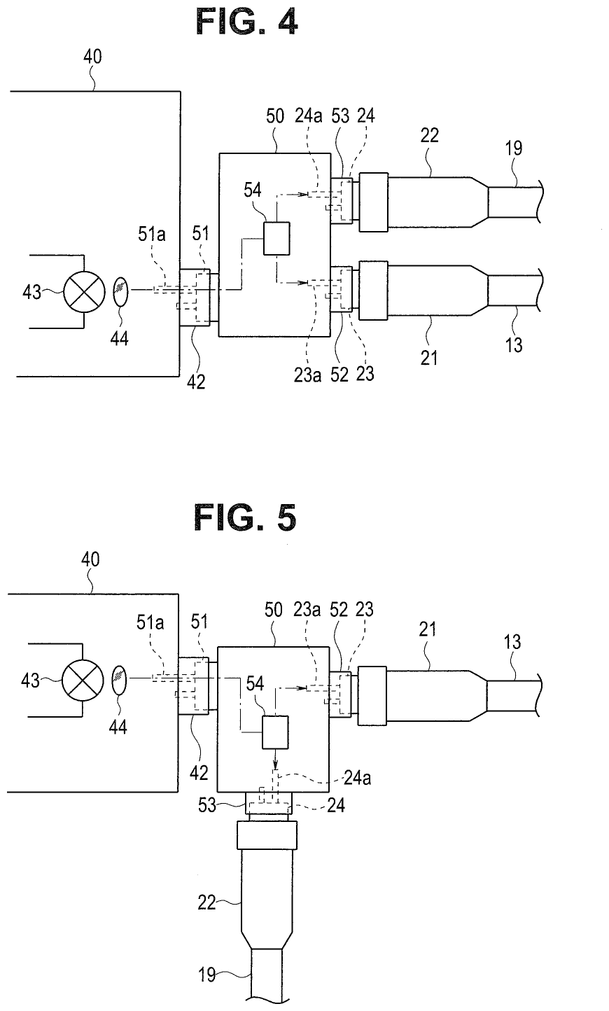

FIG. 1 illustrates an endoscope system according to one aspect of the present invention, and is an exterior view schematically illustrating a state where a baby endoscope is inserted into a mother endoscope. FIG. 2 is a plan view illustrating a state where two endoscope connectors are to be connected to a video system through a converter. FIG. 3 is a plan view illustrating a state where the two endoscope connectors are connected to the video system through the converter. FIG. 4 is a schematic view illustrating an optical distributor in the converter and a light source in the video system.

Hereinafter, description will be made on the overall configuration of an endoscope system 100 as a pancreaticobiliary endoscope system (mother-baby endoscope system) which is an endoscope system according to one aspect of the present invention, with reference to FIG. 1.

As illustrated in FIG. 1, the endoscope system 100 includes a mother endoscope 1 which is a first endoscope, a baby endoscope 2 which is a second endoscope, a video system 40 which is a camera control unit including functions of a light source apparatus and a video processor, a monitor 30 which is a display apparatus for displaying an endoscopic image, and a converter 50 which is a relay adapter.

First, the mother endoscope 1 in the endoscope system 100 includes an insertion portion 3, and an operation portion 4 provided continuously with the proximal end side of the insertion portion 3. The insertion portion 3 includes, in the following order from the distal end side, a distal end portion main body 5, a bending portion 6, and a flexible tube portion 7 that are provided continuously.

The insertion portion 3 includes, inside thereof, a treatment instrument insertion channel 8. The treatment instrument insertion channel 8 passes through the insertion portion from an operation portion side opening 9 provided on the operation portion 4 to the distal end portion main body 5. The distal end portion main body 5 includes, inside thereof, image pickup means, illumination means, etc., that are not illustrated.

The treatment instrument insertion channel 8 allows, for example, not only the insertion of the treatment instrument or other instruments but also the insertion of an insertion portion 10 of the baby endoscope 2 to be described later.

The operation portion 4 of the mother endoscope 1 includes various kinds of operation members required for operating the mother endoscope 1. A universal cable 13 is extended from the operation portion 4.

A first endoscope connector 21 is provided at the extension end of the universal cable 13. The first endoscope connector 21 is connectable to the video system 40 directly or through the converter 50. The video system 40 is an external apparatus in which an image processing apparatus that controls the mother endoscope 1, a light source apparatus, and the like are incorporated.

The operation members provided to the operation portion 4 includes, for example, an operation lever 14 for operating a treatment instrument raising stand, not illustrated, disposed at the distal end portion main body 5. In addition to the operation lever 14, the operation portion 4 includes operation switches for operating the functions of the endoscope, a bending operation knob for performing bending operation of the bending portion 6, and the like.

The bending portion 6 of the mother endoscope 1 is bendable from substantially the linear state, which is a neutral position, in desired directions, for example, in four directions, i.e., up, down, left, and right in the present embodiment. Note that the up and down directions in which the bending portion 6 is bent are set so as to be coincident with the up and down directions of the image obtained by the image pickup means of the mother endoscope 1.

Next, the baby endoscope 2 in the endoscope system 100 includes the insertion portion 10 configured to be insertable in the treatment instrument insertion channel 8 of the mother endoscope 1, and the operation portion 15 provided continuously with the proximal end side of the insertion portion 10.

The insertion portion 10 of the baby endoscope 2 is formed to have an outer diameter of about 3 to 4 mm and a length of about 2 mm, for example, so as to be insertable into the treatment instrument insertion channel 8 of the mother endoscope 1. The insertion portion 10 includes, in the following order from the distal end side, a distal end portion main body 16, a bending portion 17, and a flexible tube portion 18 that are provided continuously.

In addition, the baby endoscope 2 includes a universal cord 19 extended from the operation portion 15, and a second endoscope connector 22 provided at the extension end of the universal cord 19. Note that the second endoscope connector 22 of the baby endoscope 2 is, similarly to as the endoscope connector of the mother endoscope 1, connectable to the video system 40 directly or through the converter 50.

The main part of the insertion portion 10 of the baby endoscope 2 includes, in the following order from the distal end side, the rigid distal end portion main body 16, the bending portion 17 provided continuously with the proximal end of the distal end portion main body 16, and the flexible tube portion 18 provided continuously with the proximal end of the bending portion 17.

The distal end portion main body 16 includes, inside thereof, image pickup means, illumination means, etc., which are not illustrated. In addition, the bending portion 17 of the insertion portion 10 is bendable in a plurality of directions. The flexible tube portion 18 has flexibility and is formed to be elastic.

The bending portion 17 is bendable from substantially the linear state, which is the neutral position, in desired directions, for example, in two directions, i.e., up and down in the present embodiment. Note that the up and down directions in which the bending portion 17 is bent is set so as to be coincident with the up and down directions of the image obtained by the image pickup means of the baby endoscope 2.

The bending operation of the bending portion 17 is performed by a bending operation lever 15a provided on the operation portion 15. Note that the bending direction of the bending portion 17 is not limited to the two directions, i.e., the up and down directions, but may be four directions, i.e., the up, down, left, and right directions. Furthermore, the bending portion 17 may have various configurations, for example, the bending direction of the bending portion 17 may be two directions, i.e., left and right directions, or may be only up direction, or four directions or more.

The video system 40 includes a front panel, as a front face portion, on which an operation panel 41 and an apparatus-side receptacle portion 42 are disposed side by side in the horizontal direction (transverse direction of the video system). The operation panel 41 is a touch panel, a sheet switch, or others, through which various operations are performed, and the apparatus-side receptacle portion 42 is configured such that the first endoscope connector 21 and the second endoscope connector 22 are simultaneously connected to the receptacle portion through the converter 50.

Note that the operation panel 41 and the apparatus-side receptacle portion 42 are not limited to the configuration in which the panel and the receptacle portion are disposed side by side in the horizontal direction, but may be aligned longitudinally in the vertical direction (lengthwise direction) of the front panel of the video system 40, or may be disposed on a position other than the front panel.

The configuration of the video system 40 is the same as the conventional existing configuration. Therefore, other detailed descriptions will be omitted.

In the present embodiment, the first endoscope connector 21 of the mother endoscope 1 and the second endoscope connector 22 of the baby endoscope 2 are connectable to the one video system 40 through the converter 50, as illustrated in FIG. 2 and FIG. 3.

The video system 40 includes the apparatus-side receptacle portion 42 which is an apparatus-side connector. An adapter-side plug portion 51 of the converter 50 is connectable to the apparatus-side receptacle portion 42.

The adapter-side plug portion 51 of the converter 50 includes a light guide connector 51a and a plurality of electrical terminal portions 51b. The light guide connector 51a is extended from the front face portion of the plug portion 51 and the electrical terminal portions 51b are disposed on the outer surface of the plug portion 51.

The apparatus-side receptacle portion 42 includes a receiving connector to which the light guide connector 51a is connected by insertion and electric contacts configured to be electrically connected with the plurality of electrical terminal portions 51b (neither 51a nor 51b are illustrated).

Note that the apparatus-side receptacle portion 42 is connectable also with the first endoscope connector 21 of the mother endoscope 1 and the second endoscope connector 22 of the baby endoscope 2.

The converter 50 includes, on another surface opposite to one surface on which the adapter-side plug portion 51 is provided, a first adapter-side receptacle portion 52 which is a first receptacle portion and a second adapter-side receptacle portion 53 which is a second receptacle portion.

A first plug portion 23 of the first endoscope connector 21 is connectable to the first adapter-side receptacle portion 52, and a second plug portion 24 of the second endoscope connector 22 is connectable to the second adapter-side receptacle portion 53.

The first plug portion 23 and the second plug portion 24 respectively include light guide connectors 23a, 24a and a plurality of electrical terminal portions 23b, 24b. The light guide connectors 23a, 24a are extended respectively from the front face portions of the first and second plug portions 23, 24, and the plurality of electrical terminal portions 23b, 24b are disposed respectively on the outer surfaces of the first and second plug portions 23, 24.

In addition, the first adapter-side receptacle portion 52 includes: a receiving connector to which the light guide connector 23a is connected by insertion; and electric contacts to which the plurality of electrical terminal portions 23b are electrically connected (neither are illustrated). The second adapter-side receptacle portion 53 includes: a receiving connector to which the light guide connector 24a is connected by insertion; and electric contacts to which the plurality of electrical terminal portions 24b are electrically connected (neither are illustrated).

As illustrated in FIG. 4, the video system 40 includes, inside thereof, a light source section 43 such as a halogen lamp, an HID lamp, or an LED for illumination light, and an optical system 44 such as a lens for condensing the illumination light from the light source section 43.

The illumination light from the video system 40 is distributed to two systems by an optical distributor 54 such as an optical coupler, an optical splitter, or a prism, in the converter 50, and the distributed illumination lights are guided to the mother endoscope 1 and the baby endoscope 2 respectively through the first endoscope connector 21 and the second endoscope connector 22.

Note that each of the mother endoscope 1 and the baby endoscope 2 includes inside thereof a light guide (not illustrated) through which the illumination light is transmitted to each of the distal end portion main body 5 of the insertion portion 3 and the distal end portion main body 16 of the insertion portion 10. Then, the illumination light is applied to a subject from an illumination window (not illustrated) disposed on each of the distal end portion main bodies 5, 16.

Furthermore, though not illustrated here, the mother endoscope 1 and the baby endoscope 2 are also electrically connected with the video system 40, through the converter 50, and various kinds of control signals and image pickup signals are transmitted and received between the endoscopes and the video system.

As described above, the endoscope system 100 according to the present embodiment is configured such that a plurality of endoscopes, i.e., the mother endoscope 1 and the baby endoscope 2 are connectable simultaneously to the single existing video system 40, through the converter 50.

With such a configuration, the endoscope system 100 is capable of performing inspection and treatment on the biliary tract and the pancreatic duct, without preparing two video systems 40.

In addition, the user is not required to purchase a new video system including a function for simultaneously connecting with the mother endoscope 1 and the baby endoscope 2. As a result, the endoscope system 100 is capable of reducing unnecessary expenses, which leads to a cost reduction.

Furthermore, the endoscope system 100 requires only one video system 40, which solves the problem that the working space in the examination room is compressed by the video system.

As understood from the description above, the endoscope system 100 is capable of using a plurality of endoscopes, that is, the mother endoscope 1 and the baby endoscope 2 in the present embodiment by using the one existing video system 40.

Note that the above-described description exemplifies the endoscope system 100 in which the mother endoscope 1 and the baby endoscope 2 that perform inspection and treatment on the biliary tract and the pancreatic duct are connectable to the one video system 40. However, the endoscope system 100 is not limited to the example, and can be applied to a configuration in which a plurality of surgical laparoscopes are connectable to the one video system 40, for example.

Furthermore, the number of the endoscopes is not limited to two. If three or more adapter-side receptacle portions are provided on the converter 50, the number of endoscopes can be increased as many as the number of the adapter-side receptacle portions.

### MODIFIED EXAMPLES

The endoscope system 100 may have configurations according to the various modified examples as exemplified below.

### First Modified Example

FIG. 5 relates to the first modified example, and is a plan view illustrating the state where the two endo scope connectors are connected to the video system through the converter.

As illustrated in FIG. 5, the converter 50 may be configured such that the first adapter-side receptacle portion 52 and the second adapter-side receptacle portion 53 are disposed not on the same surface but on different surfaces of the converter 50, and the first endoscope connector 21 and the second endoscope connector 22 are connected to the converter 50 so as to face in different directions.

In the present modified example, the first adapter-side receptacle portion 52 is disposed on the other surface which is opposite side of the surface on which the adapter-side plug portion 51 is disposed, and the second adapter-side receptacle portion 53 is disposed on a side surface orthogonal to the other surface.

### Second Modified Example

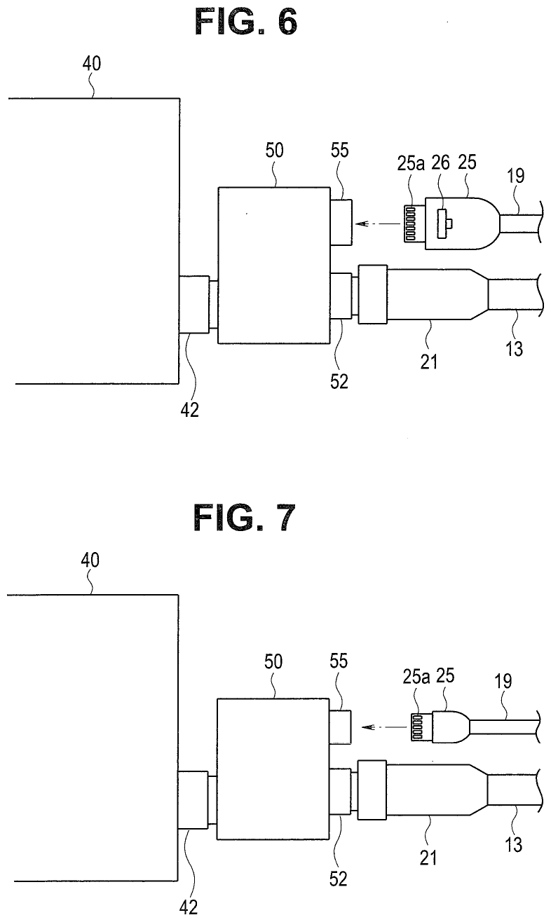

FIG. 6 relates to the second modified example, and is a plan view illustrating the state where one of the two endoscope connectors is an electrical connector that incorporates a light source, and the two endoscope connectors are to be connected to the video system through the converter.

As illustrated in FIG. 6, a second endoscope connector 25 of the baby endoscope 2 may be an electrical connector including an electrical terminal portion 25a and may incorporate inside thereof an LED 26 which is a light source of the illumination light. The converter 50 includes an electrical connector portion 55 to which the second endoscope connector 25 is connectable.

Such a configuration enables the size reduction of the second endoscope connector 25. Alternatively, the first endoscope connector 21 of the mother endoscope 1 may be an electrical connector that incorporates the LED 26.

### Third Modified Example

FIG. 7 relates to the third modified example, and is a plan view illustrating the state where one of the two endoscope connectors is an electrical connector and the two endoscope connectors are to be connected to the video system through the converter.

As illustrated in FIG. 7, a light source such as an LED, not illustrated, may be incorporated in the operation portion 4, and the second endoscope connector 25 of the baby endoscope 2 may be configured as the electrical connector. Such a configuration enables further size reduction of the endoscope connector 25. Also in the present modified example, the first endoscope connector of the mother endoscope 1 may be the electrical connector.

### Fourth Modified Example

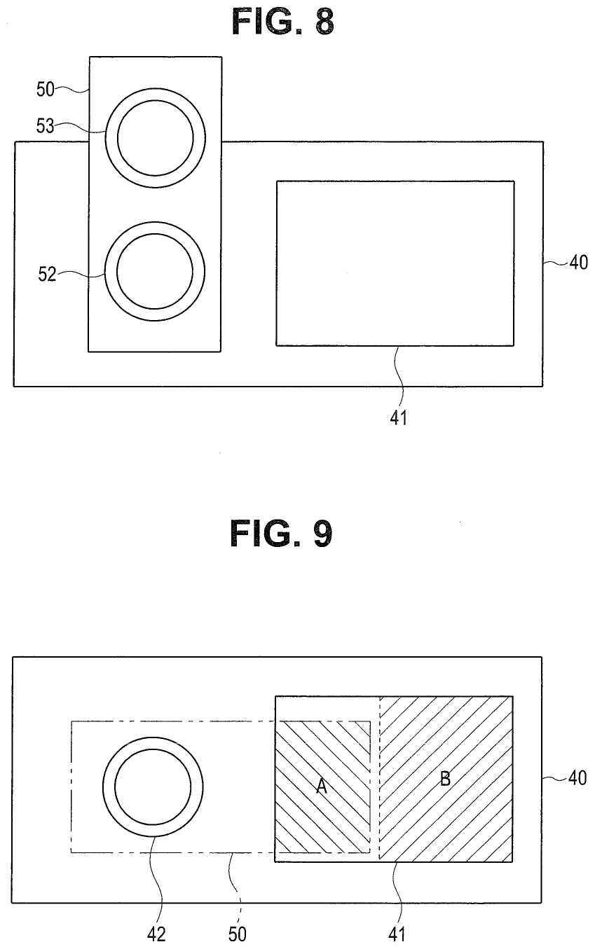

FIG. 8 relates to the fourth modified example and is a front view illustrating the state where the converter is connected to the video system.

As illustrated in FIG. 8, the operation panel 41 and the apparatus-side receptacle portion 42 of the video system 40 are provided side by side in the horizontal direction. Therefore, the converter 50 is connected to the video system 40, with the longitudinal direction of the converter being along the lengthwise direction of the video system such that the first adapter-side receptacle portion 52 and the second adapter-side receptacle portion 53 are aligned longitudinally in the vertical direction.

If the operation panel 41 and the apparatus-side receptacle portion 42 of the video system 40 are arranged longitudinally in the vertical direction, though not illustrated in the drawing, the converter 50 may be connected to the video system 40, with the longitudinal direction of the converter being along the transverse direction of the video system such that the first adapter-side receptacle portion 52 and the second adapter-side receptacle portion 53 are disposed side by side in the horizontal direction in the state where the converter 50 is connected to the video system 40.

Such a configuration allows the converter 50 to be connected to the video system 40 without covering and interfering with the operation panel 41, which prevents the operability of the operation panel 41 from being degraded.

### Fifth Modified Example

FIG. 9 relates to the fifth modified example and is a front view illustrating the state where the converter is connected to the video system.

As shown in FIG. 9, the converter 50 may be connected to the video system 40, with the longitudinal direction of the converter being along the transverse direction of the video system 40 such that the first adapter-side receptacle portion 52 and the second adapter-side receptacle portion 53 are disposed side by side in the horizontal direction.

Such a configuration creates a region A where the converter 50 covers and interferes with the operation panel 41, which causes a state where only the plurality of switches located in a region B on the operation panel 41 can be operated, the region B being a region with no interference by the converter 50.

Therefore, the switches that are not operable due to the interference by the converter 50 are switched to an operation-invalidated state so as not to respond, and the operation-invalidated switches are substituted by the plurality of switches in the region B, or operable substituted switches are mounted on the converter 50.

When the operation panel 41 is a touch panel, the layout of the switches may be changed to display all of the switches in the region B by reducing the display sizes of the switches.

### Sixth Modified Example

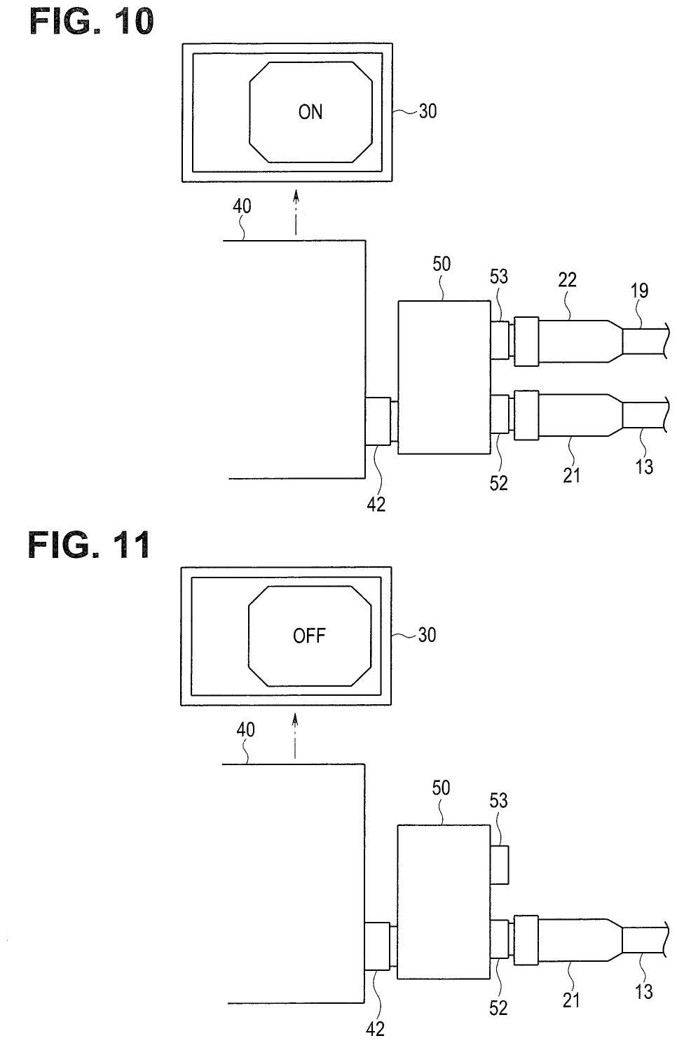

FIG. 10 relates to the sixth modified example, and is a plan view illustrating the state where the monitor and the two endoscope connectors are connected to the video system through the converter. FIG. 11 relates to the sixth modified example, and is a plan view illustrating the state where the monitor and the first endoscope connector are connected to the video system through the converter.

As illustrated in FIG. 10, the monitor 30 may be controlled to display an endoscopic image in the state where the first endoscope connector 21 of the mother endoscope 1 and the second endoscope connector 22 of the baby endoscope 2 are connected to the video system 40 through the converter 50, on the other hand, as illustrated in FIG. 11, the monitor 30 may be controlled not to display an endoscopic image in the state where only the first endoscope connector 21 is connected to the video system 40 through the converter 50.

Whether or not to display the endoscopic image on the monitor 30 is controlled by a control section (not illustrated) incorporated in the video system 40, and determination on whether to display the endoscopic image is made depending on whether power is supplied to the first endoscope connector 21 and the second endoscope connector 22.

Alternatively, in the present modified example, notification means such as an alarm, display means may be provided for notifying the state where the second endoscope connector 22 is not connected.

Even in the state where the first endoscope connector 21 is not connected, and only the second endoscope connector 22 is connected to the video system 40 through the converter 50, the same control as described above is performed.

### Seventh Modified Example

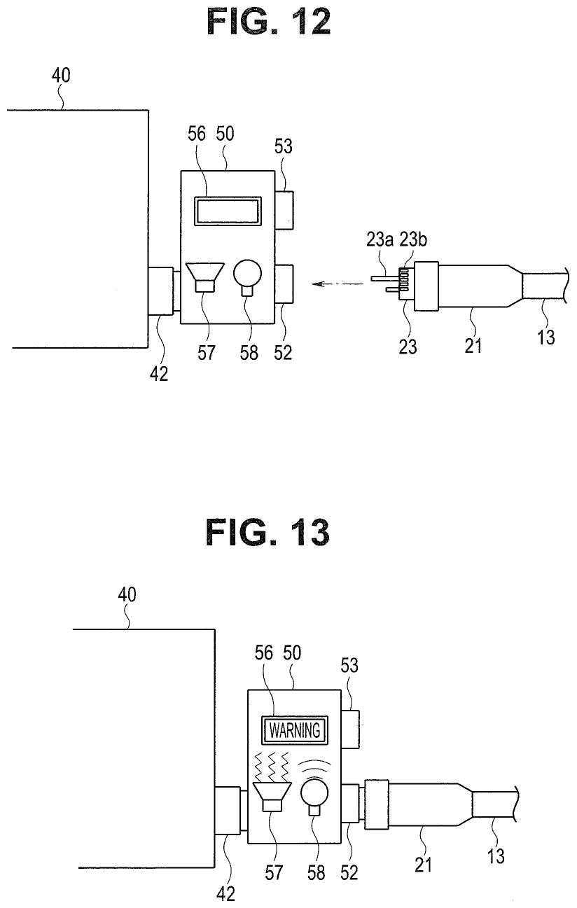

FIG. 12 relates to the seventh modified example, and is a plan view illustrating the state where the first endoscope connector is connected to the video system through the converter and the converter is provided with notification means. FIG. 13 relates to the seventh modified example, and is a plan view illustrating the state where only the first endoscope connector is connected to the video system through the converter.

As illustrated in FIG. 12, the converter 50 may be provided with the notification means such as a liquid crystal display section 56, a buzzer 57, a vibration motor 58, and a user may be notified that the system is not in the normal-use state where a plurality of endo scopes are used.

In the present modified example, as illustrated in FIG. 13, when the first endoscope connector 21 (including various endoscope connectors of various types of endoscopes) is connected, with the converter 50 connected to the video system 40 and only the mother endoscope 1 (including various endoscopes such as a front-view endoscope) is used, there is a concern that the use of the converter 50 causes influences such as decrease of the light amount of the illumination light and deterioration of the endoscopic image quality.

When one endoscope is used instead of the plurality of endoscopes, i.e., the mother endoscope 1 and the baby endoscope 2, there is no need for using the converter 50.

In the present modified example, when a user tries to use the system in the state where only the endoscope connector (the first endoscope connector 21 in the present modified example) is connected to the converter 50, characters for warning are displayed on the liquid display section 56, the buzzer 57 sounds, or the vibration motor 58 vibrates to notify the user that the converter 50 is not necessary.

### Eighth Modified Example

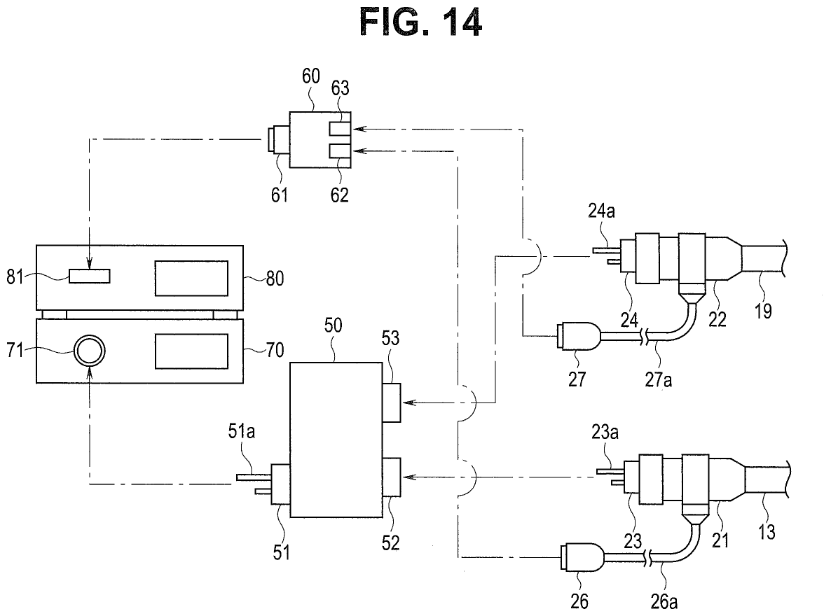

FIG. 14 relates to the eighth modified example, and is a plan view illustrating the state where two endoscope connectors and an electrical connector are to be connected respectively to a light source apparatus and a video processor through two converters.

As illustrated in FIG. 14, the first endoscope connector 21 and the second endoscope connector 22 do not include electrical terminal portions at the plug portions 23, 24. The electric cables 26a, 27a are extended and the first and second electrical connectors 26, 27 are provided respectively at the extension ends of the electric cables 26a, 27a

In addition, the endoscope system 100 includes, as separated bodies, an existing light source apparatus 70 and an existing video processor 80, which are external apparatuses, instead of the video system 40.

Note that the light source apparatus 70 includes, on the front panel thereof as the front face portion, an apparatus-side receptacle portion 71 as the first apparatus-side receptacle portion to which an adapter-side plug portion 51 of the converter 50 is connectable.

The electrical connectors 26, 27 are electrically connectable to the video processor 80 through an electrical converter 60.

The converter 60 is connectable to an apparatus-side receptacle portion 81 as a second apparatus-side receptacle provided on the front panel which is the front face portion of the video processor 80. The converter 60 includes an electrical plug portion 61, a first electrical receptacle portion 62, and a second electrical receptacle portion 63.

The first electrical connector 26 of the mother endoscope 1 is connectable to the first electrical receptacle portion 62 and the second electrical connector 27 of the baby endoscope 2 is connectable to the second electrical receptacle portion 63.

Thus, the first endoscope connector 21 and the second endoscope connector 22 are connected to the light source apparatus 70 through the converter (optical converter) 50 as a first converter, and the first electrical connector 26 and the second electrical connector 27 are connected to the video processor 80 through the converter (electrical converter) 60 as a second converter.

With the configuration of the endoscope system 100 as described above, the endoscope system 100 is capable of using the plurality of endoscopes, i.e., the mother endoscope 1 and the baby endoscope 2 in the present modified example with the one existing light source apparatus 70 and one existing video processor 80, through the two converters 50, 60.

### Ninth Modified Example

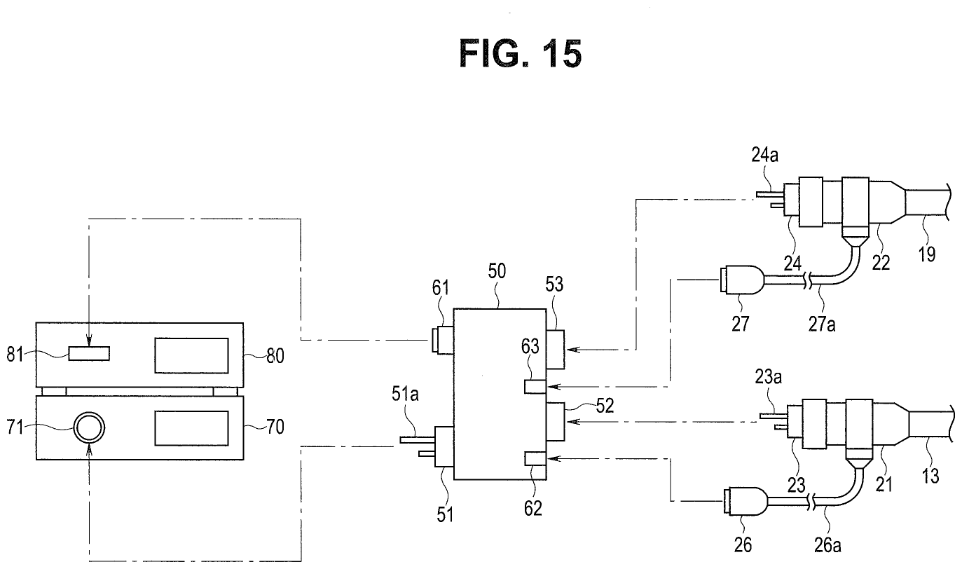

FIG. 15 relates to the ninth modified example, and is a plan view illustrating the state where two endoscope connectors and an electrical connector are to be connected to a light source apparatus and a video processor, through one converter.

As illustrated in FIG. 15, the converter 50 may be provided with the electrical plug portion 61, the first electrical receptacle portion 62, and the second electrical receptacle portion 63, and the mother endoscope 1 and the baby endoscope 2 may be connected to the one light source apparatus 70 and the one video processor 80 through the one converter 50.

The converter 50 in the present modified example is configured such that the positions of the adapter-side plug portion 51 and the electrical plug portion 61 are set such that the adapter-side plug portion 51 and the electrical plug portion 61 are connectable respectively to the apparatus-side receptacle portion 71 of the light source apparatus 70 and the apparatus-side receptacle portion 81 of the video processor 80 placed on the light source apparatus 70.

If the above-described various kinds of converters 50, 60 are for medical use, the converters have a washable, disinfectable, and sterilizable structure in order to avoid the risk of secondary infection.

The present invention as described above is not limited to the above-described embodiment and modified examples, and various changes and modifications are possible in a range without changing the gist of the present invention.

The present invention is capable of providing an endoscope system that enables a plurality of endoscopes to be used with one existing video system.

What is claimed is:

1. An endoscope system comprising:
a first endoscope comprising a first endoscope connector;
a second endoscope comprising a second endoscope connector;
an external apparatus comprising an apparatus-side receptacle to which the first endoscope connector and the second endoscope connector are connectable; and
a converter comprising a plug connectable to the apparatus-side receptacle, a first receptacle to which the first endoscope connector is connectable, and a second receptacle to which the second endoscope connector is connectable.

2. The endoscope system according to claim 1, comprising a mother-baby endoscope in which the first endoscope includes a channel and the second endoscope includes an insertion portion configured to be insertable in the channel.

3. The endoscope system according to claim 1, wherein
the external apparatus comprises a light source, and
the converter comprises an optical distributor configured to distribute light from the light source to the first endoscope connector and the second endoscope connector.

4. The endoscope system according to claim 1, wherein
the external apparatus includes a front panel on which the apparatus-side receptacle and an operation panel are disposed side by side in a horizontal direction, and
the converter is arranged so as not to overlap and interfere with the operation panel, with the first receptacle and the second receptacle aligned longitudinally in a vertical direction, and the plug of the converter is connected to the apparatus-side receptacle of the external apparatus, with a longitudinal direction of the converter being along a lengthwise direction of the external apparatus.

5. An endoscope converter comprising:
a plug connectable to an apparatus-side receptacle of an external apparatus;
a first receptacle to which a first endoscope connector included in a first endoscope is connectable; and
a second receptacle to which a second endoscope connector included in a second endoscope is connectable.

6. The endoscope converter according to claim 5, wherein
the apparatus-side receptacle of the external apparatus has a shape connectable with the first endoscope connector included in the first endoscope and the second endoscope connector included in the second endoscope.

7. An endoscope converter comprising:
a first converter-side connector to which a first endoscope-side connector included in a first endoscope is connectable;
a second converter-side connector to which a second endoscope-side connector included in a second endoscope is connectable; and
a third converter-side connector connectable to an apparatus-side connector of an external apparatus.

8. The endoscope converter according to claim 7, wherein
the apparatus-side connector of the external apparatus has a shape connectable with the first endoscope-side connector included in the first endoscope and the second endoscope-side connector included in the second endoscope.
